(19) United States
(12) Patent Application Publication
(10) Pub. No.: US 2006/0057085 A1
(43) Pub. Date: Mar. 16, 2006

(54) Anhydrous care or make-up composition containing fibers and polyols

(75) Inventor: Nathalie Jager Lezer, Bourg La Reine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 11/182,948

(22) Filed: Jul. 18, 2005

Related U.S. Application Data
(63) Continuation of application No. 09/576,546, filed on May 22, 2000, now abandoned.

(30) Foreign Application Priority Data
May 20, 1999 (FR) 99 06411

(51) Int. Cl. A61K 8/81

(52) U.S. Cl. 424/63

(57) ABSTRACT

An anhydrous care or make-up composition for keratin substances such as the skin, the lips or superficial body growths, containing fibers which are compatibilized with a fatty phase by at least one polyol that is liquid at room temperature, wherein this composition is, in particular, a foundation or a lipstick having properties of staying power and water resistance; the invention also relates to the use of fibers in a care or make-up composition for keratin substances, to give it staying power over time, in particular, at pressure and while rubbing.

The present invention relates to an anhydrous composition containing fibers, intended for cosmetics and dermatology. The invention especially applies to the care and/or treatment and/or making up of keratin substances, such as the skin, including the scalp, the lips and superficial body growths such as the eyelashes, the eyebrows, the nails and the hair of human beings.

This composition can be, in particular, in the form of a product cast as a stick or a dish, such as lipsticks or lip balms, cast foundations, concealer products, eyeshadows or face powders, in the form of a paste or a more or less fluid cream, such as fluid foundations or lipsticks, eyeliners, mascaras, care compositions, antisun compositions or coloring compositions for the skin, make-up compositions for the body or make-up compositions for the hair.

It is known to use fibers in make-up products, in particular, for their lengthening effects in mascaras (see Japanese patent application JP-A-57/158 714, the disclosure of which is incorporated herein by reference), their “textile” feel (see Japanese patent application JP-A-7/196 440, the disclosure of which is incorporated herein by reference), their fabric effect or their moisturizing properties in lip sticks (see U.S. Pat. No. 5,498,407, the disclosure of which is incorporated herein by reference) or to improve the contours of lipsticks on the edges of the lips (see European patent application EP-A-0 106 762, the disclosure of which is incorporated herein by reference). Unfortunately, it is very difficult to disperse fibers in compositions homogeneously and without forming lumps, which, in a colored composition and, in particular, in a make-up composition, generally gives a non-uniform make-up effect that is not particularly aesthetic. In addition, this difficulty of dispersion leads to compositions whose cosmetic properties are not constant and not reproducible, thus entailing problems of industrial manufacture and high manufacturing costs.

Make-up or care products for the skin or the lips of human beings, such as foundations or lipsticks, generally contain fatty phases, such as waxes and oils, pigments and/or fillers and optionally additives such as cosmetic or dermatological active agents.

When they are applied to the skin or the lips, these compositions have the drawback of transferring, i.e., of becoming at least partly deposited, leaving traces on certain supports with which they may come into contact, in particular, a glass, a cup, a cigarette, an item of clothing or the skin. This drawback results in mediocre persistence of the film applied, making it necessary regularly to repeat the application of the foundation or lipstick composition. Moreover, the appearance of these unacceptable marks, in particular, on shirt collars, can inhibit certain women from using this type of make-up.

Furthermore, these compositions have a tendency to migrate, i.e., to travel inside the wrinkles and fine lines of the skin that surround the lips and the eyes, on which these compositions are applied, resulting in an unaesthetic effect. The term “migration” means an overflowing of the composition, in particular, of the color, beyond the initial line applied.

In Japanese patent application JP-A-61 65809, the disclosure of which is incorporated herein by reference, the company Shiseido describes “transfer-resistant” lip compositions containing a siloxysilicate resin (with a three-dimensional network), a volatile silicone oil containing a cyclic silicone chain and pulverulent fillers. Moreover, the company Noevier describes, in Japanese patent application JP-A-62 61911, the disclosure of which is incorporated herein by reference, “transfer-resistant” lip composition, eyeliner and foundation compositions comprising one or more volatile silicones combined with one or more hydrocarbon-based waxes.

Although these compositions have improved “transfer-resistant” properties, they have the drawback of leaving on the lips, after the silicone oils have evaporated off, a film that becomes uncomfortable over time (sensation of drying out and of tautness), which puts a certain number of women off this type of lipstick.

In addition, these compositions based on volatile silicone oils and on silicone resins lead to matt colored films. The fact of the matter is, however, that women are nowadays looking for glossy products, in particular, for coloring the lips. Furthermore, the “transfer-resistant” properties of the films deposited are not perfect. In particular, a pronounced pressure or rubbing leads to a decrease in the color of the deposit and to redeposition onto the support placed in contact with these films.

There is thus a need for a composition that does not have the above drawbacks, and, in particular, that can have good properties of staying power and of “transfer resistance,” even when pronounced pressure or rubbing is applied, a more or less glossy appearance, that matches the desire of consumers, that does not migrate, does not dry out the skin or the lips onto which it is applied, either during the application or over time, and that gives a homogeneous, aesthetic make-up or care result.

The subject of the invention is, precisely, a care and/or treatment and/or make-up composition for keratin substances that makes it possible to overcome these drawbacks. Surprisingly, the inventor has found that the use of fibers in a make-up composition can give the composition good properties of staying power, of transfer resistance and of non-migration, while at the same time can be comfortable to wear and can have a satiny glossy appearance, and that the use of polyols can make it possible to disperse these fibers homogeneously in the composition, and thus to give a homogeneous and harmonious make-up result.

The invention applies not only to make-up products for the skin, both of the human face and body, and the lips, but also to make-up products for superficial body growths, such as the eyelashes, eyebrows, nails and hair, as well as care and/or treatment products for the skin, including the scalp.

More specifically, a subject of the invention is an anhydrous care or make-up composition for keratin substances, comprising fibers that are compatibilized with a fatty phase by means of at least one polyol that is liquid at room temperature.

The expression “anhydrous composition” should be understood as meaning a composition comprising a homogeneous continuous fatty phase in which may be dispersed ingredients that are insoluble in the fatty phase, in the absence of surfactant or emulsifier, such as dyestuffs and cosmetic or dermatological active agents, including water. In particular, the water will be present in a content of not more than 6% relative to the total weight of the composition, and, for example, less than 2% and, better still, less than 0.5%.

The expression “fatty phase” should be understood as comprising a non-aqueous, water-imiscible medium comprising one or more fatty substances chosen from compounds comprising at least 10 carbon atoms and, better still, 16 carbon atoms, silicone compounds, fluoro compounds and mixtures thereof. The organic solvents conventionally used in nail varnishes are not considered as fatty substances.

The term “fiber” should be understood as meaning an object of length L and diameter D such that L is very much greater (>>) than D, D being the diameter of the circle in which the cross section of the fiber is inscribed. In particular, the ratio L/D (or shape factor) is chosen in the range extending from 3.5 to 2500, preferably from 5 to 500 and, better still, from 5 to 150, in other words, L≧3.5 D to L≦2500 D, L≧5 D to L≦500 D, and L≧5 D to L≦150 D.

The expression “room temperature” should be understood as being a temperature of 25° C., at normal atmospheric pressure (760 mmHg).

The term “polyol” should be understood as being any organic molecule comprising at least two free hydroxyl groups. In particular, the polyol(s) of the invention has (have) an IOB (inorganic/organic balance) value of greater than 0.5, preferably greater than or equal to 0.6 and, in particular, ranging from 1 to 7 and, more especially, from 1.5 to 5.5.

The IOB parameter is known to those skilled in the art from a certain number of publications such as the article by A. Fujita, Pharm. Bull 2, 163-173 (1954) and documents JP 9-151 109 (Japanese published patent application), JP 8-217 639 (Japanese published patent application) from Shiseido or JP 9-175 925 (Japanese published patent application) from Kose, the disclosures of each of which are incorporated herein by reference.

Examples of polyols that satisfy the preceding criteria, and that can be used alone or as a mixture in the composition of the invention include:

| Name | IOB Value |
|---|---|
| Propylene glycol | 3.333 |
| Butylene glycol | 2.500 |
| Isoprene glycol | 2.222 |
| Pentylene glycol | 2.000 |
| Hexylene glycol | 1.818 |
| PEG-4 (*) | 2.656 |
| PEG-6 | 2.396 |
| PEG-8 | 2.266 |
| Glycerol | 5.000 |
| Panthenol | 3.125 |

(*) In general, polyethylene glycols (PEG) containing from 4 to 8 ethylene glycol units may be mentioned.

Examples of polyols that do not satisfy the preceding criterion relating to the IOB include:

| Name | IOB Value |
|---|---|
| PPG-10 butanediol | 0.588 |
| Polyglyceryl 3-diisostearate | 0.511 |
| Castor oil | 0.404 |

The term “compatibilization” means a total or partial solubilization or a dispersion, that is homogeneous by optical microscope, of the fibers in the fatty phase.

The fibers can be introduced into the composition of the invention according to several processes:
They can be dissolved or dispersed in one or more polyols according to the invention, and the mixture obtained can then be dissolved or dispersed, in the absence of an emulsifier, in the fatty phase of the composition, which is liquid or which has been made liquid by heating, if necessary (on condition that it is only heated to a temperature below the melting point or softening point of the fibers);
the polyol(s) according to the invention can be first dissolved or dispersed in the fatty phase of the composition, and the fibers can then be dissolved or dispersed in the polyols/fatty phase mixture.

Preferably, the first mode of incorporation of the fibers is used, using a 50/50 by weight mixture of polyols and fibers. The mixture can be prepared using a turbomixer supplying sufficient mechanical energy to wet the fibers completely, such as the Turbotest-Rayneri turbomixer sold by the company VMI (Montaigu, France).

The polyol(s) that compatibilize(s) the fibers with the fatty phase can be present in an amount ranging from 0.1 to 95% relative to the total weight of the composition, and, better still, in an amount ranging from 1 to 50%.

This composition can be used without further modification or incorporated into a more complex composition. It is, in particular, non-tacky to the touch, non-greasy and soft when applied, spreading well while at the same time having a homogeneous appearance.

The fibers that can be used in the composition of the invention may be fibers of synthetic or natural, mineral or organic origin. They may be short or long, individual or organized, for example, plaited hollow or solid. These fibers, as long as they satisfy the L/D relationship described above, can have any shape and, in particular, a circular or polygonal, e.g., square, hexagonal or octagonal, cross section depending on the specific application envisaged. In particular, their ends are blunt and/or smooth to prevent injury.

In particular, the fibers have a length ranging from 1 nm to 20 mm, preferably from 10 nm to 5 mm and, better still, from 0.1 mm to 1.5 mm. Their cross section can be within a circle of diameter ranging from 2 nm to 100 μm, preferably ranging from 20 nm to 20 μm and, better still, from 500 nm to 20 μm. The weight or yarn count of the fibers is often given in denier or decitex and represents the weight in grams per 9 km of yarn. Preferably, the fibers according to the invention have a yarn count chosen in the range extending from 0.15 to 30 deniers and, better still, from 0.18 to 18 deniers.

In order to obtain a glossy make-up result, which is most particularly desired for making up the nails and the lips, short fibers, in particular, having a length of from 1 nm to 200 μm are advantageously used. On the other hand, for a matt make-up effect, which is especially desired for making up the face (in particular, for a powder or a foundation), long fibers, in particular, being more than 200 μm long are preferably used.

The fibers can be those used in the manufacture of textiles and, in particular, silk fiber, cotton fiber, wool fiber, flax fiber, cellulose fibers extracted, in particular, from wood, from plants or from algae, polyamide (NYLON®) fiber, rayon fiber, viscose fiber, acetate fiber, in particular, rayon acetate fiber, cellulose fiber or silk fiber, poly-p-phenylene terephthamide fiber, especially KEVLAR®, acrylic fiber, in particular, polymethyl methacrylate fiber or poly(2-hydroxyethyl methacrylate) fiber, polyolefin fiber, and, in particular, polyethylene or polypropylene fiber, glass fiber, silica fiber, aramide fiber, carbon fiber, especially in graphite form, polytetrafluoroethylene TEFLON® fiber, insoluble collagen fiber, polyester fiber, polyvinyl chloride or polyvinylidene chloride fiber, polyvinyl alcohol fiber, polyacrylonitrile fiber, chitosan fiber, polyurethane fiber, polyethylene phthalate fiber, or fibers formed from a mixture of polymers, such as those mentioned above, for instance, polyamide/polyester fibers.

It is also possible to use the fibers used in surgery, such as resorbable synthetic fibers prepared from glycolic acid and ε-caprolactone, e.g., Monocryl from Johnson & Johnson; resorbable synthetic fibers such as the copolymer of lactic acid and glycolic acid, e.g., Vicryl from Johnson & Johnson; terephthalic polyester fibers, e.g., Ethibond from Johnson & Johnson; and stainless steel threads, e.g., Acier from Johnson & Johnson, in particular, for use in nail varnishes.

Moreover, the fibers can be coated or uncoated and surface-treated or otherwise. Examples of coated fibers that can be used in the invention include polyamide fibers coated with copper sulphide for an antistatic effect, for example, R-STAT from Rhodia, or another polymer allowing a particular organization of the fibers (specific surface treatment) or surface treatment which induces color/hologram effects, for example, Lurex fiber from Sildorex.

Preferably, fibers of synthetic origin are used, and, in particular, organic fibers, such as those used in surgery.

The fibers that can be used in the composition according to the invention are preferably polyamide or poly-p-phenylene terephthamide fibers. Their length (L) can range from 0.1 to 5 mm, preferably from 0.25 to 1.6 mm, and their average diameter (D) can range from 5 to 50 μm. In particular, the polyamide fibers sold by the company P. Bonte under the name Polyamide 0.9 Dtex 0.3 mm, having an average diameter of 6 μm, a weight of about 0.9 dtex and a length ranging from 0.3 mm to 1.5 mm, can be used. Poly-p-phenylene terephthamide fibers with an average diameter of 12 μm and a length of about 1.5 mm, such as those sold under the name Kevlar Floc by the company Du Pont Fibers, can also be used.

The fiber concentration depends on the specific application and on the type of product envisaged. For a facial make-up product, such as a foundation, or a product for the lips, such as a lipstick, the fiber concentration can range from 0.1 to 20% relative to the total weight of the composition, preferably from 0.5 to 10%. For a special effect, in particular, for making up the body, the nails or the hair, the amount of fibers can be up to 30% relative to the total weight of the composition.

The composition of the invention can be in the form of a paste, a solid, a more or less fluid cream or even an anhydrous lotion. It can be a rigid or supple anhydrous gel, optionally cast as a stick or dish. Preferably, it is in cast form.

The fatty phase of the composition of the invention can contain one or more fatty substances that are liquid at room temperature and atmospheric pressure, known as oils. These oils can be hydrocarbon-based oils of animal, plant, mineral or synthetic origin, silicone oils and/or fluoro oils, and mixtures thereof.

The expression “hydrocarbon-based oil” means an oil mainly containing carbon atoms and hydrogen atoms and, in particular, alkyl or alkenyl chains, such as alkanes or alkenes, but also an oil containing an alkyl or alkenyl chain comprising one or more ether, ester or carboxylic acid groups.

Particular examples of oils that can be used in the composition according to the invention include:
hydrocarbon-based oils of animal origin, such as perhydrosqualene;
hydrocarbon-based plant oils, such as liquid triglycerides of fatty acids of 4 to 10 carbon atoms, such as heptanoic or octanoic acid triglyceride, or alternatively sunflower oil, corn oil, soybean oil, marrow oil, grapeseed oil, sesame oil, hazelnut oil, apricot oil, macadamia oil, arara oil, castor oil, avocado oil, caprylic/capric acid triglycerides, such as those sold by the company Stearineries Dubois or those sold under the names Miglyol 810, 812 and 818 by the company Dynamit Nobel, jojoba oil or karite butter;
linear or branched hydrocarbons of mineral or synthetic origin, such as liquid paraffin and derivatives thereof, petroleum jelly, polydecenes and hydrogenated polyisobutene, such as parleam;
synthetic esters and ethers, in particular, of fatty acids, such as the oils of formula R₁COOR₂ in which R₁ represents a higher fatty acid residue containing from 7 to 29 carbon atoms and R₂ represents a hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyidodecyl erucate, isostearyl isostearate; hydroxylated esters such as isostearyl lactate, octyl hydroxystearate, octyldodecyl hydroxystearate, diisostearyl malate, triisocetyl citrate, fatty alkyl heptanoates, octanoates or decanoates; polyol esters such as propylene glycol dioctanoate, neopentyl glycol diheptanoate, diethylene glycol diisononanoate; and pentaerythritol esters such as pentaerythrityl tetraisostearate;
fatty alcohols containing from 12 to 26 carbon atoms, such as octyldodecanol, 2-butyloctanol, 2-hexyldecanol, 2-undecylpentadecanol and oleyl alcohol;
partially hydrocarbon-based and/or silicone-based fluoro oils;
silicone oils, such as volatile or non-volatile, linear or cyclic polydimethyl siloxanes (PDMSs); polydimethyl siloxanes comprising alkyl, alkoxy or phenyl groups, pendant or at the end of the silicone chain, these groups containing from 2 to 24 carbon atoms; phenylsilicones, such as phenyl trimethicones, phenyl dimethicones, phenyl trimethylsiloxydiphenylsiloxanes, diphenyl dimethicones, diphenylmethyl-diphenyltrisiloxanes and 2-phenylethyltrimethylsiloxysilicates,
mixtures thereof.

These oils can represent from 0.2 to 99.85% relative to the total weight of the composition, preferably from 1 to 80% and, better still, from 10 to 80%.

The composition of the invention can also comprise any ingredient usually used in the field under consideration, such as dyestuffs, for instance pigments, nacres, dyes that are soluble in the liquid fatty phase or in the polyols according to the invention, antioxidants, essential oils, preserving agents, fragrances, liposoluble polymers, in particular, hydrocarbon-based liposoluble polymers, such as polyalkylenes or polyvinyl laurate, agents for gelling the liquid fatty phase, agents for structuring the liquid fatty phase, such as waxes, gums and fillers, pigment-dispersing agents, cosmetic or dermatological active agents, such as, for example, emollients, moisturizers (water), vitamins, liquid lanolin, essential fatty acids, lipophilic or hydrophilic sunscreens and mixtures thereof. These ingredients can be present in the composition in the amounts usually used in the fields under consideration.

Needless to say, a person skilled in the art will take care to select the optional additional ingredients and/or the amount thereof such that the advantageous properties of the composition according to the invention are not, or are not substantially, adversely affected by the addition envisaged.

Advantageously, the composition contains waxes to rigidify it. The waxes can be hydrocarbon-based waxes, fluoro waxes and/or silicone waxes and can be of plant, mineral, animal and/or synthetic origin. In particular, the waxes have a melting point of greater than 45° C.

Examples of waxes that can be used in the invention include hydrocarbon-based waxes, i.e., containing only carbon and hydrogen atoms, silicone waxes and/or fluoro waxes, optionally comprising ester, hydroxyl or thiol functions. By way of example, mention may be made of lanolin, beeswax, carnauba wax, candelilla wax, paraffin, lignite wax or microcrystalline wax, ceresin or ozokerite; synthetic waxes, such as polyethylene waxes, silicone waxes, such as alkyl or alkoxydimethicone containing from 16 to 45 carbon atoms, Fischer-Tropsch waxes and mixtures thereof.

The nature and amount of the waxes depends on the desired mechanical properties and textures. As a guide, the composition can contain from 0 to 50% by weight of waxes, relative to the total weight of the composition, and, better still, from 5 to 30%. These waxes are also structuring agents for the composition.

The composition of the invention can advantageously comprise a particulate phase, generally present in a proportion of from 0 to 40% relative to the total weight of the composition, preferably from 0.5 to 25% and, better still, from 1 to 25% and that can comprise pigments and/or nacres and/or fillers usually used in cosmetic or dermatological compositions.

The term “pigments” should be understood as meaning white or colored, mineral or organic particles that are insoluble in the liquid fatty phase and are intended to color and/or opacify the composition. The term “fillers” should be understood as meaning colorless or white, mineral or synthetic, lamellar or non-lamellar particles. The terms “nacres” should be understood as meaning iridescent particles, in particular, produced by certain molluscs in their shell or else synthesized. These fillers and nacres serve, in particular, to modify the texture of the composition and, in particular, form part of the structuring agents capable of leading to a solid form.

The pigments can be present in the composition in a proportion of from 0.05 to 25% relative to the weight of the final composition, and preferably in a proportion of from 2 to 15%. Examples of mineral pigments that can be used in the invention include titanium oxide, zirconium oxide or cerium oxide, as well as zinc oxide, iron oxide or chromium oxide and ferric blue. Examples of organic pigments that can be used in the invention include carbon black and barium or strontium lakes, calcium lake (DC Red No. 7) or aluminium lake (DC Red No. 21 or FDC Yellow No. 6).

The nacres can be present in the composition in a proportion of from 0 to 20% relative to the total weight of the composition, preferably in a content of about 1 to 15%. Examples of nacres that can be used in the invention include mica coated with titanium oxide, with iron oxide, with natural pigment or with bismuth oxychloride, such as colored titanium mica.

The fillers can be present in a proportion of from 0 to 35% relative to the total weight of the composition, preferably 0.5 to 15%. Particular examples include talc, mica, kaolin, NYLON (in particular ORGASOL) powder and polyethylene powder, TEFLON, starch, boron nitride, copolymer microspheres such as EXPANCEL (Nobel Industry), POLYTRAP (Dow Corning) and silicone resin microbeads (for example, TOSPEARL from Toshiba).

The composition according to the invention can be in the form of a dermatological or skin care composition or in the form of an antisun or make-up-removing composition. In this case, it can be present in uncolored form, optionally containing cosmetic or dermatological active agents. It can then be used as a care base for the skin or the lips (lip balms for protecting the lips against cold weather and/or sunlight and/or the wind) or a day or night care cream.

The composition of the invention can also be in the form of a colored make-up product for the skin, in particular, a foundation, a blusher, a face powder, an eyeshadow or a make-up for the lips, such as a lipstick or a lip gloss, optionally having care or treatment properties, or, alternatively, in the form of an eyeliner, mascara or nail varnish.

The invention can also be in the form of a treating or non-treating, dyeing or non-dyeing shampoo or a conditioner product.

Needless to say, the composition of the invention should be cosmetically or dermatologically acceptable, i.e., non-toxic, and able to be applied to the skin or the lips as well as to the superficial body growths of human beings.

The composition according to the invention can be manufactured by the known processes generally used in cosmetics or dermatology.

A subject of the invention is also a cosmetic care or treatment process for keratin substances, and, in particular, the skin or the lips of human beings, comprising the application to keratin substances of the composition, in particular, the cosmetic composition, as defined above.

Another subject of the invention is the cosmetic use of fibers in an anhydrous care or make-up composition for keratin substances, containing a fatty phase and at least one polyol that is liquid at room temperature, to give the composition staying power over time, in particular, at pressure and while rubbing, and/or gloss.

Another subject of the invention is a cosmetic process to impart staying power over time and/or gloss to an anhydrous care or make-up composition containing a fatty phase and at least one polyol that is liquid at room temperature, this process comprising introducing fibers into the composition.

The present invention is further illustrated by the following examples which are designed to teach those of ordinary skill in the art how to practice the invention. The following examples are merely illustrative of the invention and should not be construed as limiting the invention as claimed.

### EXAMPLE 1

### Lipstick Containing Polyamide Fibers

| | |
|---|---|
| Polyethylene wax | 7% |
| Microcrystalline wax | 7% |
| Sesame oil | 24% |
| Jojoba oil | 24% |
| Phenyltrimethicone | 3% |
| Polyamide 0.9 Dtex fiber (0.3 mm long | 5% |
| and having an average diameter of 6 μm) | |
| Pigments | 9.5% |
| Lanolin | 10.5% |
| Glycerol | 10% |

Preparation: The fibers were introduced into the glycerol at room temperature (25° C.) and the mixture was stirred at 1000 rpm for 30 min with a Rayneri turbomixer. Separately, the mixture of waxes and oils was heated to a temperature of 100° C., followed by introduction of the pigments into the molten mixture, with stirring in a three-roll turbomixer. The glycerol/fiber mixture was introduced into the wax/oil/pigments mixture. The combined mixture was mixed in a vessel (at 100° C.) with magnetic stirring for 20 min. The mixture was then cast in a mold preheated to 45° C., to give a stick.

A glossy lipstick was obtained that has good properties of staying power and resistance to rubbing and to pressure, providing a homogeneous and aesthetic make-up result. In particular, the transfer-resistant properties of this lipstick were compared with those of a lipstick according to the prior art containing the same ingredients except for the fibers, the fiber content being compensated for by the glycerol content. The application of these two compositions was carried out comparatively per half-lip. After drying in ambient air for 2 min, the test users placed a “kiss” on a sheet of printing paper. They reapplied lipstick per half-lip and then placed a “kiss” on the sheet of paper after drying in ambient air for 10 min. Another “kiss” was placed on the sheet of paper after drying in ambient air for 50 min. The assessment of the transfer resistance was visual.

100% of the test users noted very much superior transfer-resistant properties for the lipstick according to the invention compared with that of the prior art, for the three “kiss” tests.

### EXAMPLE 2

### Anhydrous Foundation Containing Polyamide Fibers

| | |
|---|---|
| Microcrystalline wax | 3% |
| Camauba wax | 5.5% |
| Octyl palmitate | 27.5% |
| Octyldodecanol | 10% |
| Polyamide 0.9 Dtex fibers (0.3 mm long | 3% |
| and having an average diameter of 6 μm) | |
| Glycerol | 7% |
| Pigments | 10% |
| Nanometric TiO₂ | 9% |
| Magnesium silicate | 8% |
| Nylon powder | 9.5% |
| Silica powder | 4% |
| Active agents | 2.5% |

This foundation was prepared in the same way as the lipstick in Example 1, with introduction of the active agents last, followed by casting in a suitable container. The foundation obtained gave a homogeneous, aesthetic, soft, non-greasy make-up result that did not transfer even under pressure.

### EXAMPLE 3

### Lipstick Containing Kevlar Floc Fibers

| | |
|---|---|
| Oxypropylenated beeswax | 14.5% |
| Microcrystalline wax | 3% |
| Oxypropylenated lanolin wax | 2% |
| Sesame oil | 18% |
| Arara oil | 10% |
| Oleyl erucate | 10% |
| Lanolin | 20% |
| Acetylated lanolin | 6% |
| Kevlar fibers (1.5 mm long and having | 2% |
| an average diameter of 12 μm) | |
| Propylene glycol | 7% |
| Pigments | 10.5% |

This stick of lipstick was prepared in the same way as the lipstick of Example 1. The lipstick obtained gave a homogeneous, aesthetic, soft, non-greasy make-up result that did not transfer even under pressure.

### EXAMPLE 4

### Lipstick Containing Polyamide Fibers

| | |
|---|---|
| Polyglycerolated (3 mol) beeswax | 4.4% |
| Microcrystalline wax | 11% |
| Oxypropylenated lanolin wax | 6.2% |
| Sesame oil | 12.4% |
| Arara oil | 12.4% |
| Oleyl erucate | 12.4% |
| Lanolin | 20% |
| Acetylated lanolin | 6.2% |
| Polyamide 0.9 Dtex fibers (0.3 mm long | 5% |
| and having an average diameter of 6 μm) | |
| Pigments | qs 100% |

This stick of lipstick was compared with a stick of lipstick of the same composition in which the 5% of fibers were replaced with 5% of a spherical powder of copolymer of lauryl methacrylate/ethylene glycol methacrylate (Polytrap 6603 sold by APS), and was judged to be less glossy than the lipstick of the invention by a panel of experts. The Polytrap powder has a diameter D of 0.1 to 1 μm and a shape factor L/D of 0.

The foregoing written description relates to various embodiments of the present invention. Numerous changes and modifications may be made therein without departing from the spirit and scope of the invention as defined in the following claims.

What is claimed is:

1. An anhydrous care or make-up composition comprising fibers, wherein said fibers are compatibilized with a fatty phase by at least one polyol, further wherein said at least one polyol is liquid at room temperature, and wherein said fibers have a length much greater (>>) than their diameter.

2. A composition according to claim 1, wherein said at least one polyol has an IOB value ranging from 1 to 7.

3. A composition according to claim 2, wherein said at least one polyol has an IOB value ranging from 1.5 to 5.5.

4. A composition according to claim 1, wherein said at least one polyol is chosen from propylene glycol, butylene glycol, isoprene glycol, pentylene glycol, hexylene glycol, polyethylene glycols containing from 4 to 8 ethylene glycol units, glycerol and panthenol.

5. A composition according to claim 1, wherein said at least one polyol is present in an amount ranging from 0.1 to 95% relative to the total weight of the composition.

6. A composition according to claim 5, wherein said at least one polyol is present in an amount ranging from 1 to 50% relative to the total weight of the composition.

7. A composition according to claim 1, wherein said fibers are chosen from silk fiber, cotton fiber, wool fiber, flax fiber, cellulose fibers extracted from wood, from plants or from algae, polyamide fiber, rayon fiber, viscose fiber, acetate fiber, cellulose fiber or silk fiber, poly-p-phenylene terephthamide fiber, acrylic fiber, polyolefin fiber, glass fiber, silica fiber, aramide fiber, carbon fiber, polytetrafluoroethylene TEFLON® fiber, insoluble collagen fiber, polyester fiber, polyvinyl chloride or polyvinylidene chloride fiber, polyvinyl alcohol fiber, polyacrylonitrile fiber, chitosan fiber, polyurethane fiber, polyethylene phthalate fiber, fibers formed from a mixture of polymers, and surgical fibers.

8. A composition according to claim 7, wherein said fibers are cellulose fibers extracted from wood.

9. A composition according to claim 7, wherein said acetate fiber is rayon acetate fiber.

10. A composition according to claim 7, wherein said acrylic fiber is chosen from polymethyl methacrylate fiber and poly(2-hydroxyethyl methacrylate) fiber.

11. A composition according to claim 7, wherein said polyolefin fiber is chosen from polyethylene and polypropylene fiber.

12. A composition according to claim 7, wherein said carbon fiber is in a graphite form.

13. A composition according to claim 1, wherein said fibers are fibers of synthetic origin.

14. A composition according to claim 1, wherein said fibers are polyamide fibers or poly-p-phenylene terephthamide fibers.

15. A composition according to claim 1, wherein said fibers have a length L and a diameter D wherein L/D is chosen in a range extending from 3.5 to 2500.

16. A composition according to claim 15, wherein said L/D is chosen in a range extending from 5 to 500.

17. A composition according to claim 16, wherein said L/D is chosen in a range extending from 5 to 150.

18. A composition according to claim 1, wherein said fibers have a length ranging from 0.1 to 5 mm.

19. A composition according to claim 18, wherein said fibers have a length ranging from 0.25 to 1.5 mm.

20. A composition according to claim 1, wherein said fibers have an average diameter ranging from 5 to 50 μm.

21. A composition according to claim 1, wherein said fibers have a yarn count ranging from 0.15 to 30 deniers.

22. A composition according to claim 21, wherein said fibers have a yarn count ranging from 0.18 to 18 deniers.

23. A composition according to claim 1, wherein said fatty phase comprises at least one oil that is liquid at room temperature, chosen from hydrocarbon-based oils of animal, plant or mineral origin, silicone oils and fluoro oils.

24. A composition according to claim 1, further comprising at least one oil chosen from perhydrosqualene; heptanoic and octanoic acid triglycerides, sunflower oil, corn oil, soybean oil, marrow oil, grapeseed oil, sesame oil, hazelnut oil, apricot oil, macadamia oil, arara oil, castor oil, avocado oil, caprylicicapric acid triglycerides, jojoba oil and karite butter; liquid paraffin and derivatives thereof, petroleum jelly, polydecenes and hydrogenated polyisobutene; purcellin oil, isopropyl myristate, isononyl isononanoate, 2-ethylhexyl palmitate, 2-octyldodecyl stearate, 2-octyldodecyl erucate, isostearyl isostearate; isostearyl lactate, octyl hydroxystearate, octyidodecyl hydroxystearate, diisostearyl malate, triisocetyl citrate, fatty alkyl heptanoates, octanoates and decanoates; propylene glycol dioctanoate, neopentyl glycol diheptanoate, diethyelene glycol diisononanoate; pentaerythritol esters; octyldoddecanol, 2-butyloctanol, 2-hexyldecanol, 2-undecylpentadecanol and oleyl alcohol; volatile or non-volatile, linear or cyclic polymethyl siloxanes that are liquid at room temperature; phenyl trimethicones, phenyltrimethylsiloxydiphenylsiloxanes, diphenyl dimethicones, diphenylmethyldiphenyltrisiloxanes, and 2-phenylethyltrimethylsiloxysilicates.

25. A composition according to claim 24, wherein said hydrogenated polyisobutene is parleam.

26. A composition according to claim 1, further comprising at least one particulate filler.

27. A composition according to claim 1, wherein said composition is in the form of a rigid or supple anhydrous gel, optionally cast as a stick or dish.

28. A composition according to claim 1, further comprising at least one ingredient chosen from dyestuffs, antioxidants, essential oils, preserving agents, fragrances, liposoluble polymers, agents for gelling the liquid fatty phase, waxes, gums, fillers, dispersants, and cosmetic and dermatological active agents.

29. A lipstick or a lip gloss, a foundation, a concealer product, a mascara, an eyeliner, a face powder, an eyeshadow, a make-up product for the body, an antisun care product or a make-up product for the hair comprising fibers, wherein said fibers are compatibilized with a fatty phase by at least one polyol, further wherein said at least one polyol is liquid at room temperature, and wherein said fibers have a length much greater (>>) than their diameter.

30. A cosmetic care or treatment process for human keratin substances, comprising applying to said human keratin substances an anhydrous care or make-up composition comprising fibers, wherein said fibers are compatibilized with a fatty phase by at least one polyol, further wherein said at least one polyol is liquid at room temperature, and wherein said fibers have a length much greater (>>) than their diameter.

31. A method of improving the staying power over time and/or gloss of an anhydrous care or make-up composition containing a fatty phase and at least one polyol that is liquid at room temperature comprising including fibers in said anhydrous care or make-up composition.

32. The method according to claim 31, wherein said method improves the staying power of an anhydrous care or make-up composition while rubbing.
